(19) United States
(12) Patent Application Publication
(10) Pub. No.: US 2026/0158262 A1
(43) Pub. Date: Jun. 11, 2026

(54) CANNULA ADAPTOR FOR PERCUTANEOUS BLOOD PUMP

(71) Applicant: BOSTON SCIENTIFIC SCIMED, INC., Maple Grove, MN (US)

(72) Inventors: Brice Lee Shireman, Maple Grove, MN (US); Jeffrey Matthew Lucas, Eden Prairie, MN (US); Reggie Roth, Monticello, MN (US); Shawn Johnson, Hanover, MN (US)

(73) Assignee: BOSTON SCIENTIFIC SCIMED, INC., Maple Grove, MN (US)

(21) Appl. No.: 19/412,233

(22) Filed: Dec. 8, 2025

Related U.S. Application Data
(60) Provisional application No. 63/729,643, filed on Dec. 9, 2024.

(51) Int. Cl. A61M 60/857; A61M 60/13; A61M 60/216; A61M 60/804

(52) U.S. Cl. CPC A61M 60/857; A61M 60/13; A61M 60/216; A61M 60/804

(57) ABSTRACT

Percutaneous circulatory support devices and systems are disclosed which may include a blood pump, a flexible cannula extending distal of the blood pump, an adaptor including a plurality of struts defining a plurality of blood inflow windows therebetween. The adaptor may include a proximal end coupled to a distal end of the cannula at an interface. A distal tip may be coupled to a distal end of the adaptor and extend distally therefrom, whereby the interface between the adaptor and the distal end of the cannula may have a circular cross-sectional shape in an equilibrium configuration. The interface between the adaptor and the distal end of the cannula may be configured and/or otherwise adapted to deform when subjected to an external force during use to alter the cross-sectional shape to a non-circular cross-sectional shape.

### CROSS REFERENCE TO RELATED APPLICATIONS

This application claims the benefit of U.S. Provisional Patent Application Ser. No. 63/729,643 filed on Dec. 9, 2024, the disclosure of which is incorporated herein by reference.

### TECHNICAL FIELD

The present disclosure pertains to cannula adaptors for use with medical devices such as percutaneous circulatory support devices. More particularly, the present disclosure pertains to flexible cannula adaptors for use with a percutaneous blood pump.

### BACKGROUND

Percutaneous mechanical circulatory support devices, such as blood pumps can provide transient support for extended use in patients whose heart function or cardiac output is compromised. The percutaneous mechanical circulatory support devices may be sufficiently flexible to be navigated through the vasculature to a patient's heart. Such devices may be navigated through the aortic arch and placed across the aortic valve, for example. Various configurations of percutaneous mechanical circulatory support devices are known. However, there is an ongoing need to provide improved construction and improved materials germane to percutaneous mechanical circulatory support devices and associated components.

### BRIEF SUMMARY

This disclosure provides design, material, manufacturing method, and use alternatives for medical devices, including percutaneous circulatory support devices and associated percutaneous blood pumps.

In at least a first example, a percutaneous circulatory support system is disclosed. In this and other examples, a percutaneous circulatory support system may include a blood pump, a flexible cannula extending distal of the blood pump, an adaptor including a plurality of struts defining a plurality of blood inflow windows therebetween. The adaptor may include a proximal end coupled to a distal end of the cannula at an interface. In this and other examples, a distal tip may be coupled to the distal end of the adaptor and extend distally therefrom, whereby the interface between the adaptor and the distal end of the cannula has a circular cross-sectional shape in an equilibrium configuration, and whereby the interface between the adaptor and the distal end of the cannula may be configured and/or otherwise adapted to deform when subjected to an external force during use to alter the cross-sectional shape to a non-circular cross-sectional shape.

Alternatively or additionally to any of the examples above, the plurality of struts may extend to the proximal end of the adaptor.

Alternatively or additionally to any of the examples above, each of the plurality of struts may include a proximal face directly secured to a distal end face of the cannula.

Alternatively or additionally to any of the examples above, the proximal face of each of the plurality of struts may be coupled to the distal end face of the cannula by one or more of welding, soldering and adhesive bonding.

Alternatively or additionally to any of the examples above, the proximal end of the adaptor may include an opening formed by proximal ends of the plurality of struts.

Alternatively or additionally to any of the examples above, the plurality of struts may be configured and/or otherwise adapted to bend and/or flex independent of one another.

Alternatively or additionally to any of the examples above, the distal end of the cannula may be coupled to the proximal end of the adaptor by one or more of welding, soldering, and adhesive bonding.

Alternatively or additionally to any of the examples above, the proximal end region of the adaptor may include a bendable annulus, whereby the plurality of struts extends proximally to the bendable annulus.

Alternatively or additionally to any of the examples above, the adaptor may be formed of a nickel-titanium alloy, preferably nitinol.

In another non-limiting example, a percutaneous circulatory support system is described herein. In this and other examples, a percutaneous circulatory support system may include a blood pump, a flexible cannula extending distal of the blood pump, an adaptor including a plurality of struts defining a plurality of blood inflow windows therebetween, each of the plurality of struts including a proximal face directly secured to a distal end face of the cannula, the adaptor having a proximal end coupled to a distal end of the cannula at an interface, whereby the proximal end of the adaptor may include an opening formed by the proximal ends of the plurality of struts. In this and other examples, a distal tip may be coupled to a distal end of the adaptor and extend distally therefrom, whereby the interface between the adaptor and the distal end of the cannula has a circular cross-sectional shape in an equilibrium configuration, and whereby the interface between the adaptor and the distal end of the cannula may be configured and/or otherwise adapted to deform when subjected to an external force during use to alter the cross-sectional shape to a non-circular cross-sectional shape.

Alternatively or additionally to any of the examples above, the plurality of struts may be formed of one or more of nitinol, stainless steel, titanium and a shape memory alloy.

Alternatively or additionally to any of the examples above, the plurality of struts may extend to the proximal end of the adaptor.

Alternatively or additionally to any of the examples above, the proximal face of each of the plurality of struts may be coupled to the distal end face of the cannula by one or more of welding, soldering and adhesive bonding.

Alternatively or additionally to any of the examples above, the plurality of struts may be configured to bend and/or flex independent of one another.

Alternatively or additionally to any of the examples above, the distal end of the cannula is coupled to the proximal end of the adaptor by one or more welding, soldering, and adhesive bonding.

In other non-limiting examples, a percutaneous circulatory support system may include a blood pump, a flexible cannula extending distal of the blood pump, an adaptor formed of one or more nickel-titanium alloys and including a plurality of struts defining a plurality of blood inflow windows therebetween. Each of the plurality of struts may include a proximal face directly secured to a distal end face of the cannula, the adaptor may include a proximal end region coupled to a distal end of the cannula at an interface. In this and other examples, the proximal end region of the adaptor may include a bendable annulus, whereby the plurality of struts extends proximally to the bendable annulus. A distal tip may be coupled to a distal end of the adaptor and extend distally therefrom, whereby the interface between the adaptor and the distal end of the cannula may have a circular cross-sectional shape in an equilibrium configuration, and whereby the interface between the adaptor and the distal end of the cannula may be configured to deform when subjected to an external force during use to alter the cross-sectional shape to a non-circular cross-sectional shape.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary percutaneous circulatory support device including a percutaneous blood pump;

FIG. 2 shows the distal end region of the percutaneous circulatory support device of FIG. 1 including the percutaneous blood pump;

FIG. 3 is an example of a cannula adaptor in accordance with at least one embodiment of the present disclosure.

FIG. 4 is an enlarged view of a cannula adaptor attached to a cannula in accordance with at least one embodiment of the present disclosure.

FIG. 5 is an example of a cannula adaptor in accordance with at least one embodiment of the present disclosure.

FIG. 6 is an example of a cannula adaptor attached to a cannula in accordance with at least one embodiment of the present disclosure.

FIG. 7 is an example of a cannula adaptor in accordance with at least one embodiment of the present disclosure.

FIG. 8 is an example of a cannula adaptor in accordance with at least one embodiment of the present disclosure.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

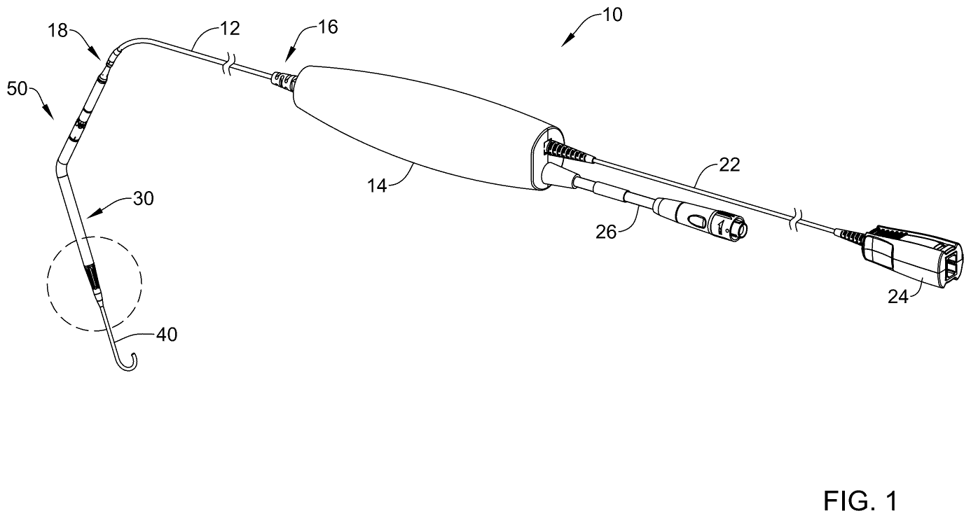

FIG. 1 illustrates a perspective view of a percutaneous circulatory support device 10 including a percutaneous blood pump 50 located at a distal end region thereof. The percutaneous circulatory support device 10 may be coupled to or include the blood pump 50, with an elongate shaft 12 of the percutaneous circulatory support device 10 extending proximally from the percutaneous blood pump 50 and a distal tip 40 extending distally from the blood pump 50. For instance, a proximal end 16 of the elongate shaft 12 may be coupled to a junction housing 14 and a distal end 18 of the elongate shaft 12 may be coupled to the percutaneous blood pump 50. An electrical cable 22 may extend from the junction housing 14 to a connector 24 at a proximal end thereof. The connector 24 may be configured to be connected to a controller (not shown) for controlling the blood pump 50, such as providing electrical power to the blood pump 50. The percutaneous circulatory support device 10 may also include an extension 26 connectable to the controller for sending and/or receiving signals, such as from one or more sensors during operation of the blood pump 50.

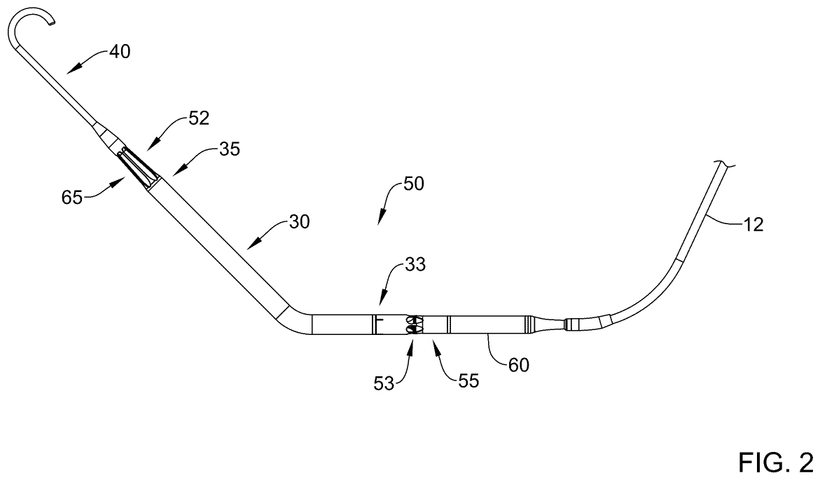

Additional features of the blood pump 50 are illustrated in FIG. 2. The blood pump 50 may generally include a flexible cannula 30, which extends from a proximal end 33 of the flexible cannula 30 to a distal end 35 of the cannula 30, an adaptor 65 extending distally of the flexible cannula 30, a distal tip 40 extending distally of the adaptor 65, an impeller housing 55, and a motor housing 60. In some embodiments, the flexible cannula 30, the impeller housing 55 and/or the motor housing 60 may be integrally or monolithically constructed. In other instances, the flexible cannula 30, the impeller housing 55 and/or the motor housing 60 may be separate components. The impeller housing 55 carries an impeller assembly therein (not shown). The impeller assembly may include an impeller, secured to an impeller shaft, that rotates relative to the impeller housing 55 to drive blood through the blood pump 50. In some embodiments, the impeller shaft and the impeller of the impeller assembly may be integrally formed, whereas, in other embodiments the impeller shaft and the impeller may be separate components.

Rotation of the impeller causes blood to flow from a blood inlet 52 of the blood pump 50, such as at and/or proximate to a distal end of the flexible cannula 30, through the flexible cannula 30 and the impeller housing 55, and out of a blood outlet 53 proximal of the impeller, such as through a sidewall formed on the impeller housing 55. In some instances, the blood inlet 52 may include a plurality of blood inlet windows (also referred to as blood inflow windows) arranged around a circumference of the blood pump 50 (e.g., the flexible cannula 30) and/or arranged around a circumference of the adaptor 65 as will be described further herein. In some instances, the blood outlet 53 may include a plurality of blood outflow windows arranged around a circumference of the impeller housing 55. In other embodiments, the blood inlet 52 and/or the blood outlet 53 may be formed on other portions of the blood pump 50.

The cannula 30 may be a flexible slotted tube having cuts or striations formed in the wall of the cannula 30 to increase flexibility of the cannula 30. In other non-limiting examples, cannula 30 may be provided with apertures and/or windows and these may be provided in addition to, or alternative to the aforementioned cuts and/or striations. In yet other instances, the cannula may be a braided tubular member, providing the cannula 30 with a desired flexibility.

With continued reference to FIG. 2, the motor housing 60 carries a motor configured to rotatably drive the impeller of the impeller assembly relative to the impeller housing 55. Electrical power may be supplied to the motor through wiring extending through the elongate shaft 12, for example. In some instances, the motor may be physically connected to the impeller. For example, in some embodiments the impeller may be mounted on the drive shaft of the motor. In other embodiments, the impeller shaft may be directly or indirectly coupled to the drive shaft of the motor. In some instances, the drive assembly may include a magnetic coupling between the motor and the impeller. For example, a driving magnet may be mounted on the drive shaft of the motor. Rotation of the driving magnet causes rotation of a driven magnet, which is connected to the impeller assembly. More specifically, in embodiments incorporating an impeller shaft, the impeller shaft and the impeller of the impeller assembly are configured to rotate with the driven magnet. In other embodiments, the motor may be coupled to the impeller assembly via other components.

The blood pump 50 may be guided over a guidewire during introduction of the blood pump 50 into the vasculature of a patient. For instance, a guidewire, inserted through a guidewire lumen (not shown), may be advanced proximally along the impeller assembly and out through one of the outflow windows of the blood outlet 53. With the guidewire tracked through the blood pump 50, the percutaneous circulatory support device 10 may be advanced over the guidewire into a vasculature.

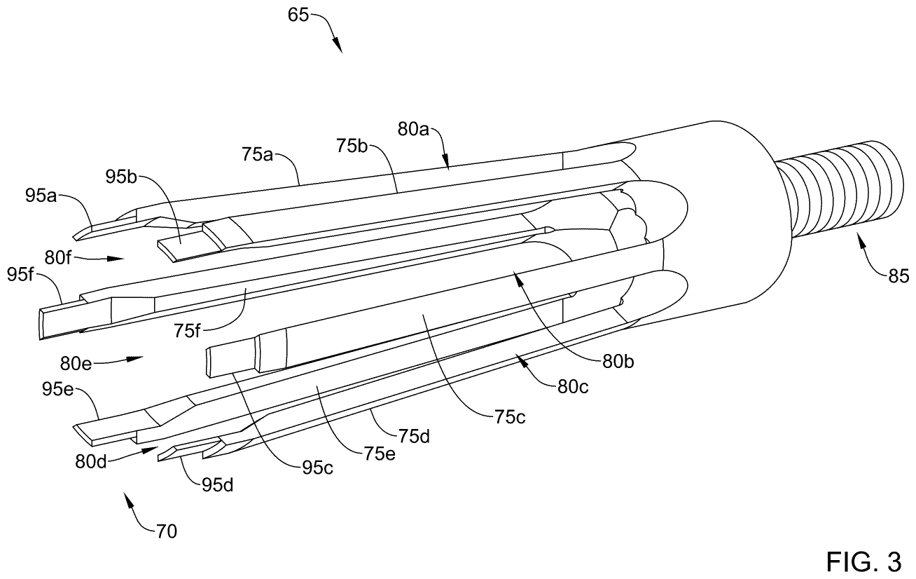

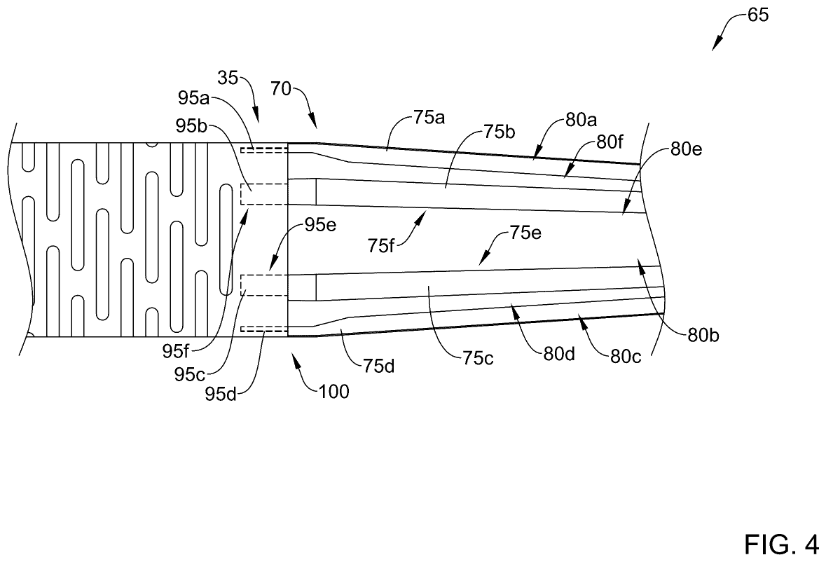

FIG. 3 shows an enlarged view of an example cannula adaptor (also referred to as “adaptor”) described herein. As shown in FIG. 3, the adaptor 65 includes a proximal end 70 configured and/or otherwise adapted to connect, couple with, weld to, bond to and/or adhere to the cannula distal end 35 (see FIG. 4) of the cannula 30. In this and other examples, the adaptor 65 may include a plurality of struts 75a-f (collectively, struts 75) defining a plurality of blood inflow windows 80a-f (collectively, blood inflow windows 80) therebetween. However, more or fewer struts 75 are additionally and/or alternatively contemplated as well as more or fewer windows 80. Struts 75a-f may extend from the adaptor proximal end 70 toward the adaptor distal end 85, which forms a connector that may attach to the distal tip 40. Thus, the struts 75a-f may extend proximally to the proximal extent of the adaptor 65 such that the proximal ends of the struts 75a-f are spaced apart from one another and unattached to other portions of the adaptor 65. In some instances, the struts 75a-f may extend a majority of the length of the adaptor 65.

The interface between the adaptor 65 and the distal end of the cannula 30 (i.e., cannula distal end 35) may be configured and/or otherwise adapted to deform when subjected to an external force during use to alter its circular or substantially circular cross-sectional shape to a non-circular cross-sectional shape. In this and other examples, the interface may be a plurality of proximal faces 95a-f of struts 75a-f, which are the proximal endpoints of the struts 75a-f and are suitable for connection, coupling, welding, soldering, adhering, and/or the like to the distal end 35 of the cannula 30. Proximal faces 95a-f may collectively form and/or conform to a circular or substantially circular geometry when viewed proximally. In other words, the collection of the ends of proximal faces 95a-f may conform to and/or trace a circular or substantially circular shape and/or outline when viewed from the proximal end of proximal faces 95a-f. Proximal faces 95a-f may be uniform in construction with the plurality of struts 75a-f (i.e., monolithic) or may be tapered, grooved, notched, and/or may include protrusions, knurls, depressions, or other like features which enable connection, coupling, welding, soldering, adhering, and/or the like to cannula distal end 35.

The plurality of blood inflow windows 80a-f may be formed in the spaces between the plurality of struts 75a-f. As each of the plurality of struts 75a-f are configured to bend and/or flex independently of each other, the spaces forming the plurality of blood inflow windows 80a-f may change and/or shift in geometry. For example, in an equilibrium condition, when the adaptor 65 is not acted upon by an external and/or outside force, the adaptor proximal end 70 may hold a constant circular shape or circular outline and the blood inflow windows 80a-f may maintain constant spacing and/or consistent geometry between each of the plurality of struts 75a-f. However, when an external and/or outside force acts on the adaptor 65 and/or one or more components of the adaptor 65, each of the plurality of struts 75a-f may bend and/or flex independent of the remainder of the plurality of struts 75a-f. Additionally, the adaptor proximal end 70 may deform, bend, and/or flex into a non-circular cross-section, a non-circular geometry, a non-circular shape, a non-circular outline, and/or a non-circular configuration. Such non-circular cross-sections, geometries, shapes, outlines and/or configurations may include, but are not limited to those that are oval, ovular, elliptical, egg-shaped, or the like.

Turning to FIG. 4, a cannula distal end 35, located in the region of the dotted circle shown in FIG. 1, is shown in accordance with multiple examples described herein connected or secured to the proximal end of the adapter 65 to define a bendable annulus 100 at the junction between the flexible cannula 30 and the adapter 65. The bendable annulus 100 may deform, bend, and/or flex from a circular cross-section or shape into a non-circular cross-section, a non-circular geometry, a non-circular shape, a non-circular outline, and/or a non-circular configuration. Such non-circular cross-sections, geometries, shapes, outlines and/or configurations may include, but are not limited to those that are oval, ovular, elliptical, egg-shaped, or the like. Cannula distal end 35 may be bonded, adhered, welded, soldered and/or otherwise coupled to a plurality of struts 75a-f of adaptor 65. The plurality of struts 75a-f may be welded to, adhered to, and/or otherwise connected to cannula distal end 35 through various processes including but not limited to: arc welding, flux-cored arc welding, plasma arc welding, electron-beam welding, laser-beam welding, atomic-hydrogen welding, electroslag welding, metal inert gas welding, tungsten inert gas welding, stick welding, gas welding, and/or any other welding process known in the art. In this and other examples, and/or in addition to welding, the plurality of struts 75a-f may be soldered to cannula distal end 35 by any soldering process known in the art.

For example, the proximal faces 95a-f of the struts 75a-f may be secured to the distal end 35 of the cannula 30. For example, the proximalmost extents of the struts 75a-f may be inserted into the lumen of the cannula 30 through the distal opening of the cannula 30 such that the proximal faces 95a-f may be secured (e.g., welded) to an inner surface of the cannula 30 and/or a distally facing annular terminal distal end surface of the cannula 30. In other instances, a proximalmost extent of the proximal faces 95a-f may abut the distally facing annular terminal distal end surface of the cannula 30 and be secured (e.g., welded) thereto.

Further shown in FIG. 4 are a plurality of blood inflow windows 80a-f which may be formed in the spaces residing between the plurality of struts 75a-f. The area (i.e., negative space between struts 75 and/or void between struts 75) of the blood inflow windows 80 may change during operation and/or use of the percutaneous circulatory systems described herein as the plurality of struts 75a-f may each independently bend and/or flex relative to one another and/or in unison with one another. As such, as each of the plurality of struts 75 may bend and/or flex, it can be appreciated that the area of the blood inflow windows 80 formed between the plurality of struts 75 may change in correspondence to the bending and/or flexion and/or alteration of the plurality of struts 75.

The struts 75 (which may also be referred to as arms, projections, and intermediate members) may also be cantilevered to one or more of the cannula 30, cannula distal end 35 and the distal tip 40 of the percutaneous circulatory support devices and/or systems disclosed herein. Further, and even in a cantilevered configuration, one or more of the cannula 30, cannula distal end 35, plurality of struts 75a-f and other components connected to and/or associated with the adaptor 65 may bend, flex, deform, and/or alter cross-sectional area and/or geometry when an outside and/or external force is applied to the aforementioned components and features.

Although the disclosure is not limited to the incorporation of nitinol as a material basis for the features disclosed herein, it has been found that nitinol and other alloyed forms of nitinol provide distinct advantages for the purposes of the disclosure defined herein. In at least one instance, it has been found that nitinol is superior to the implementation of stainless steel in avoidance of material failure as a cannula adaptor (or other like structure) is moved through an introducing element such as an introducer sheath. Further, it has been shown, at least in part due to a more flexible flexural modulus of nitinol relative to that of stainless steel, that if a kink or other obstruction in an introducing element should occur, the superior flexural modulus of nitinol allows the described nitinol-composed structures disclosed herein to bend, flex, compress, and/or otherwise preferentially conform to surroundings to avoid material failure, to avoid and prevent occlusion within an introducer device, and to further maintain structural integrity of the nitinol-formed devices herein as they are navigated through tortuous anatomy.

Components of the cannula 30, cannula distal end 35, adaptor 65, adaptor proximal end 70, adaptor distal end 85, adaptor proximal ring 90 (described further herein), and/or struts 75a-f of any of the examples disclosed herein may be formed of nitinol, nickel-titanium alloys, titanium and/or other shape memory materials and/or alloys. In other non-limiting examples, the aforementioned components and features of the present disclosure may be formed of stainless steel and/or may have some and/or all parts formed of stainless steel and/or may have some and/or all parts formed of nitinol or like shape memory materials and/or alloys. In other words, some, most, and/or all components and/or features of the cannula 30, cannula distal end 35, adaptor 65, adaptor proximal end 70, adaptor distal end 85, adaptor proximal ring 90, and/or struts 75a-f may be formed of a combination or permutation of stainless steel, nitinol, nickel-titanium alloys, titanium and/or other shape memory materials and/or shape memory alloys.

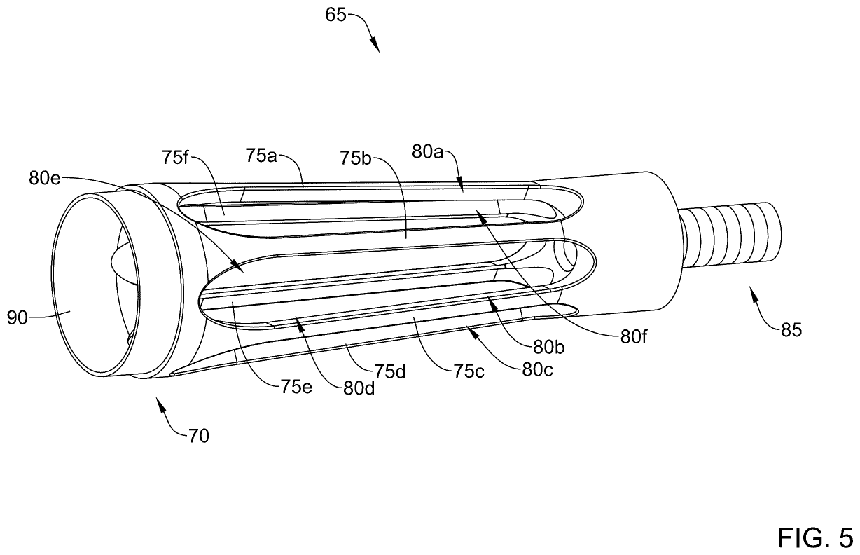

Another exemplary adaptor 65 is shown in FIG. 5. As illustrated, adaptor 65 may include an adaptor proximal ring 90 which may be formed integral with, bonded and/or adhered, and/or coupled to adaptor proximal end 70. Adaptor proximal ring 90 may be flexible, bendable, deformable and may also be referred to as an annulus, bendable annulus, deformable annulus, flexible annulus, ring, bendable ring, deformable ring, flexible ring or the like. The proximal ends of struts 75a-f may be connected to and/or otherwise coupled to adaptor proximal ring 90 and extend to adaptor distal end 85, forming a plurality of blood inflow windows 80a-f in the spaces between each of the plurality of struts 75a-f. These aforementioned components may bend, flex, and/or deform under the application of an outside and/or external force, such as that exerted when the adaptor 65 is moved through an outer covering such as an introducer sheath or like introducer element. Further, these aforementioned components may restore to their original shapes and/or geometries upon removal of an outside and/or external force, therefore returning to their original shape and/or geometry in an equilibrium state (i.e., in the absence of applied external and/or outside force or forces and/or during normal and/or ideal and/or expected usage of the adaptor and associated components and features described herein).

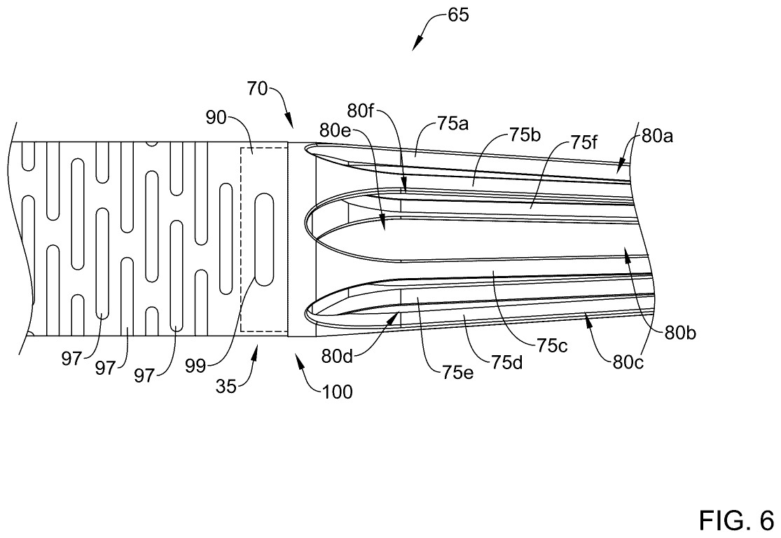

FIG. 6 shows an enlarged view of an adaptor 65 which includes an adaptor ring 90 which is shown bonded (i.e., welded) and/or otherwise coupled to cannula distal end 35, located in the region of the dotted circle shown in FIG. 1. For example, the adaptor ring 90, or at least a portion thereof, may be inserted into the lumen of the cannula 30 through the distal opening of the cannula 30 such that the adaptor ring 90 may be secured (e.g., welded) to an inner surface of the cannula 30 and/or a distally facing annular terminal distal end surface of the cannula 30. In other instances, a proximalmost extent of the adaptor ring 90 may abut the distally facing annular terminal distal end surface of the cannula 30 and be secured (e.g., welded) thereto. The distal end 35 of the flexible cannula 30 may connected or secured to the proximal end of the adapter 65 to define a bendable annulus 100 at the junction between the flexible cannula 30 and the adapter 65. The bendable annulus 100 may deform, bend, and/or flex from a circular cross-section or shape into a non-circular cross-section, a non-circular geometry, a non-circular shape, a non-circular outline, and/or a non-circular configuration. Such non-circular cross-sections, geometries, shapes, outlines and/or configurations may include, but are not limited to those that are oval, ovular, elliptical, egg-shaped, or the like.

Adaptor ring 90 may bend and/or flex in the presence of an applied external and/or outside force, such as that experienced when traversing tortuous anatomy and/or moving through an introducer element such as an introducer sheath or the like. Adaptor ring 90 may be provided with a circular or substantially circular geometry and/or cross-section in an original, equilibrium configuration, and may bend and/or flex into a non-circular geometry and/or cross-section in the presence of applied external and/or outside force and may restore to its original configuration in the absence of applied external and/or outside force and/or forces.

As shown in FIG. 6 and applicable to other examples, the flexible cannula 30 may include a tubular member having a plurality of cuts of slots 97 defined therein to provide the flexible cannula 30 with a desired flexibility. In other instances, the flexible cannula 30 may include a braided tubular member providing the flexible cannula 30 with a desired flexibility. Other configurations of the flexible cannula 30 are also contemplated. The cannula distal end 35 and/or adaptor ring 90 may further include windows and/or apertures 99, which may improve the flexibility of flexible cannula 30 and/or adaptor ring 90 among providing other beneficial features. In some instances, a weldment may be provided in apertures 99 to secure the adaptor ring 90 to the distal end 35 of the flexible cannula 30. Struts 75a-f may extend from adaptor ring 90, or alternatively, may extend from adaptor proximal end 70, or in other examples, may extend directly from cannula distal end 35. Blood inflow windows 80a-f may be formed in the spaces (i.e., voids) in between struts 75a-f and may change in area and/or geometry as struts 75a-f are able to independently flex and/or bend relative to other struts within the plurality of struts 75a-f. In other words, and applicable to other examples described herein, when struts 75a-f bend and/or flex independently to one another, the space between each strut may increase and/or decrease, and therefore the area occupied in those spaces (i.e., negative space forming blood inflow windows 80) may increase and/or decrease and thus may change in geometry.

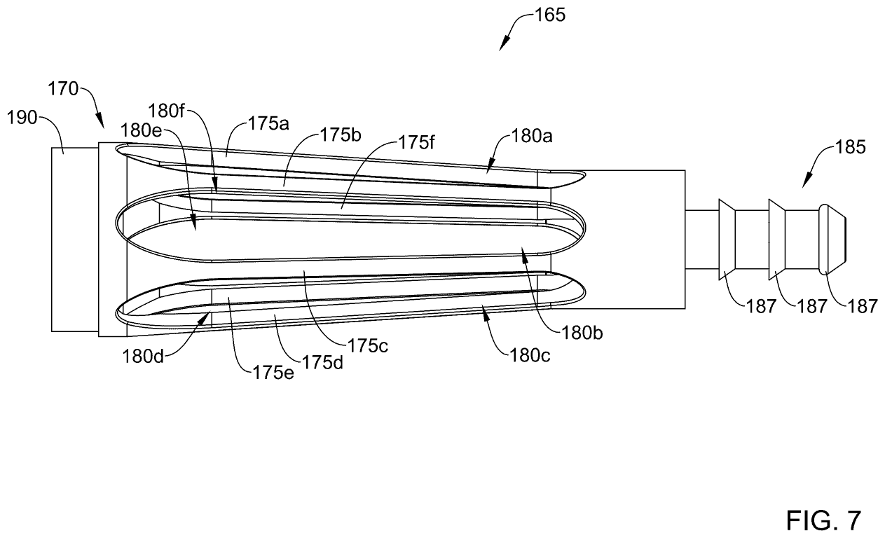

Turning to FIG. 7, another example adaptor 165 in accordance with the present disclosure is shown. Adaptor 165 may include an adaptor distal end 185 for connection to the distal tip 40 of any of the percutaneous circulatory support devices and/or systems disclosed herein. As shown in FIG. 7, and in a non-limiting sense, adaptor distal end 185 may form a connector that is barbed with barbs 187 such as illustrated in FIG. 7. However, other connector configurations for adaptor distal end 185 are contemplated, including but not limited to distally facing barbs, proximally facing barbs, orthogonal barbs, a mix of distally and proximally facing barbs, two-headed barbs (i.e., barbs with both proximal and distal facing elements and/or barbs with one or more proximal and distal facing elements and/or an orthogonal facing element), three-headed barbs (i.e., barbs with proximal, distal, and/or orthogonal facing elements) a single-barb connector, a mix of barbs and other protrusions, a connector comprising one or more protrusions, a connector comprising one or more depressions, a connector comprising one or more recesses, a connector comprising one or more grooves, a connector comprising one or more notches, a connector comprising one or more knurls, and/or any combination or permutation of the aforementioned.

FIG. 7 further demonstrates that a plurality of struts 175a-f may be connected and/or otherwise coupled to adaptor proximal ring 190 at the proximal end 170 of the adaptor 165, and may extend distally from the proximal ring 190 toward adaptor distal end 185, which may also be referred to as a connector. In this and other examples, a plurality of blood inflow windows 180a-f may be formed by the space (i.e., negative space, area, void etc.) between each of the plurality of struts 175a-f. It can be appreciated that the spacing of blood inflow windows 180a-f may change as an external force is applied to struts 175a-f which urges struts 175a-f to bend and/or flex independently of one another, and thus may also change the geometry of the blood inflow windows 180a-f. It can be appreciated that this principle is applicable to other examples disclosed herein.

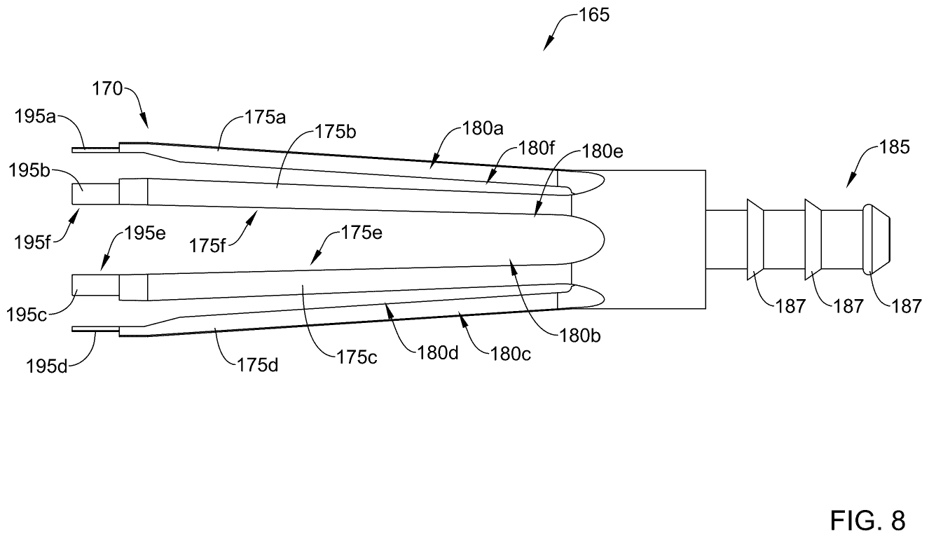

FIG. 8 illustrates another example of an adaptor 165, similar to the adaptor 65 of FIG. 3, in which the proximal end of the plurality of struts 175a-f extend to the proximal extent of the adaptor 165 and form an opening in the space outlined by the proximal faces 195a-f of the plurality of struts 175a-f. The opening of this and other examples may be circular or substantially circular in an original, equilibrium configuration, as provided in similar configurations. The opening of this and other examples may also change geometry as the struts 175a-f may bend and/or flex independently relative to one another, especially as an external force and/or external forces are applied to the adaptor 165, thus altering the cross-sectional shape and/or geometry of the struts 175a-f and proximal faces 195a-f. Proximal faces 195a-f of the plurality of struts 175a-f may be continuous with the plurality of struts 175a-f, or in other words forming a monolithic structure with the plurality of struts 175a-f. Alternatively, or additionally, proximal faces 195a-f may be bonded and/or otherwise coupled to the proximal struts 175a-f, and/or may be tapered, slotted, knurled, rounded, blunt, hollowed, curved, pointed, jagged, zig-zagged, spiral, helical, slanted, flattened, bulbous, square, triangular, polygonal, or any of the like or any combination or permutation of the aforementioned.

As shown in FIG. 8, adaptor distal end 185 may form a connector having a barbed connection including one or more barbs 187 for facilitating connection to the proximal end of the distal tip 40. However, in other non-limiting examples, the adaptor distal end 185 may include two or more barbs, three or more barbs, or four barbs or more. It is further contemplated that other structures may be implemented supplemental to, or in lieu of barbs, and may be applied to the adaptor distal end 185 as a connector for connection with the distal tip 40. Structures include, but are not limited to protrusions, grooves, knurls, notches, keys, projections, protuberances, couplings, male-female connections, ports, conduits, locks, depressions, valleys, recesses, peaks, projections and/or any combination or permutation of the aforementioned.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A percutaneous circulatory support system, comprising:
a blood pump;
a flexible cannula extending distal of the blood pump;
an adaptor including a plurality of struts defining a plurality of blood inflow windows therebetween, the adaptor having a proximal end coupled to a distal end of the cannula at an interface;
a distal tip coupled to a distal end of the adaptor and extending distally therefrom;
wherein the interface between the adaptor and the distal end of the cannula has a circular cross-sectional shape in an equilibrium configuration, and
wherein the interface between the adaptor and the distal end of the cannula is configured to deform when subjected to an external force during use to alter the cross-sectional shape to a non-circular cross-sectional shape.

2. The system of claim 1, wherein the plurality of struts extend to the proximal end of the adaptor.

3. The system of claim 1, wherein each of the plurality struts includes a proximal face directly secured to a distal end face of the cannula.

4. The system of claim 3, wherein the proximal face of each of the plurality of struts is coupled to the distal end face of the cannula by one or more of welding, soldering and adhesive bonding.

5. The system of claim 2, wherein the proximal end of the adaptor comprises an opening formed by proximal ends of the plurality of struts.

6. The system of claim 2, wherein the plurality of struts are configured to bend and/or flex independent of one another.

7. The system of claim 1, wherein the distal end of the cannula is coupled to the proximal end of the adaptor by one or more of welding, soldering, and adhesive bonding.

8. The system of claim 1, wherein a proximal end region of the adaptor comprises a bendable annulus, wherein the plurality of struts extends proximally to the bendable annulus.

9. The system of claim 8, wherein the adaptor is formed of a nickel-titanium alloy, preferably nitinol.

10. A percutaneous circulatory support system, comprising:
a blood pump;
a flexible cannula extending distal of the blood pump;
an adaptor including a plurality of struts defining a plurality of blood inflow windows therebetween, each of the plurality of struts including a proximal face directly secured to a distal end face of the cannula, the adaptor having a proximal end coupled to a distal end of the cannula at an interface;
wherein the proximal end of the adaptor comprises an opening formed by the proximal ends of the plurality of struts;
a distal tip coupled to a distal end of the adaptor and extending distally therefrom;
wherein the interface between the adaptor and the distal end of the cannula has a circular cross-sectional shape in an equilibrium configuration, and
wherein the interface between the adaptor and the distal end of the cannula is configured to deform when subjected to an external force during use to alter the cross-sectional shape to a non-circular cross-sectional shape.

11. The system of claim 10, wherein the plurality of struts extend to the proximal end of the adaptor.

12. The system of claim 10, wherein the proximal face of each of the plurality of struts is coupled to the distal end face of the cannula by one or more of welding, soldering and adhesive bonding.

13. The system of claim 10, wherein the plurality of struts are configured to bend and/or flex independent of one another.

14. The system of claim 10, wherein the distal end of the cannula is coupled to the proximal end of the adaptor by one or more of welding, soldering, and adhesive bonding.

15. The system of claim 10, wherein the proximal end of the adaptor comprises an opening formed by proximal ends of the plurality of struts.

16. The system of claim 10, wherein the plurality of struts are configured to bend and/or flex independent of one another.

17. The system of claim 10, wherein a proximal end region of the adaptor comprises a bendable annulus, wherein the plurality of struts extends proximally to the bendable annulus.

18. The system of claim 10, wherein the adaptor is formed of a nickel-titanium alloy, preferably nitinol.

19. A percutaneous circulatory support system, comprising:
a blood pump;
a flexible cannula extending distal of the blood pump;
an adaptor formed of one or more nickel-titanium alloys and including a plurality of struts defining a plurality of blood inflow windows therebetween, each of the plurality of struts including a proximal face directly secured to a distal end face of the cannula, the adaptor having a proximal end region coupled to a distal end of the cannula at an interface;
wherein the proximal end region of the adaptor comprises a bendable annulus, wherein the plurality of struts extends proximally to the bendable annulus.
a distal tip coupled to a distal end of the adaptor and extending distally therefrom;
wherein the interface between the adaptor and the distal end of the cannula has a circular cross-sectional shape in an equilibrium configuration, and
wherein the interface between the adaptor and the distal end of the cannula is configured to deform when subjected to an external force during use to alter the cross-sectional shape to a non-circular cross-sectional shape.

20. The system of claim 19, wherein the plurality of struts are configured to bend and/or flex independent of one another.
